(19) United States
(12) Patent Application Publication
(10) Pub. No.: US 2002/0019061 A1
(43) Pub. Date: Feb. 14, 2002

(54) PURIFICATION AND ANALYSIS OF CYCLIC PEPTIDE LIBRARIES, AND COMPOSITIONS THEREOF

(76) Inventors: HUNG-SEN LAI, WINCHESTER, MA (US); JIHONG WANG, MEDFORD, MA (US)

Correspondence Address: TESTA, HURWITZ & THIBEAULT, LLP, HIGH STREET TOWER, 125 HIGH STREET, BOSTON, MA, 02110, US

(21) Appl. No.: 09/387,590

(22) Filed: Aug. 31, 1999

(51) Int. Cl. C07K 5/12; C07K 5/04

(52) U.S. Cl. 436/518; 530/344; 530/345; 530/333

(57) ABSTRACT

Disclosed are methods of purifying and analyzing cyclic peptide libraries using hydrophobic-hydrophobic interaction. An embodiment of the invention includes associating a hydrophobic tag, e.g., a fluorenylmethoxycarbonyl group (Fmoc), with non-cyclized linear peptide contaminants, followed by separation of the cyclic peptide library from the tagged linear peptide contaminants using a hydrophobic support such as a reversed phase chromatography column. Consequently, the cyclic peptide library is separated from the linear peptide contaminants since the linear peptide contaminants elute later from the support. Another aspect of the invention is directed to the analysis of the proportion of linear peptide contaminants in a cyclic peptide library. Also included within the scope of the invention are the compositions of matter which result from practicing methods of the invention.

### FIELD OF THE INVENTION

The invention relates to methods of purifying and analyzing cyclic peptide libraries, and compositions thereof. More specifically, the invention relates to methods of purifying cyclic peptide libraries using hydrophobic-hydrophobic interactions, and analysis of the crude and purified cyclic peptide libraries.

### BACKGROUND

Many biological responses are mediated by the interaction of a binding protein with a target compound. The specificity of a particular binding protein, such as an enzyme, typically has been determined by identifying a number of natural substrates for the protein, obtaining sequence information of these substrates, then comparing the sequences of these substrates to define a consensus motif for substrate binding. However, there are many limitations to this approach. For example, the procedure is quite expensive and laborious since an optimal substrate sequence is not likely to be determined unless each amino acid residue within a putative binding motif is altered individually and then evaluated to determine its importance.

Consequently, alternative approaches have been developed to avoid isolating and examining the sequences of native substrates. With the advent of combinatorial synthetic chemistry and improved screening techniques, compounds of interests readily may be identified and further studied for their useful biological properties. One such approach synthesizes linear peptide libraries, then evaluates the sequence specificity of the peptide binding sites for a particular domain of interest. See, e.g., CELL (1993) 72: 767-778; and U.S. Pat. No. 5,532,167.

Another approach uses cyclic peptide libraries composed of natural and/or unnatural œ-amino acids. See, e.g., International Application No. PCT/US98/10876 (WO 98/54577). The peptide sequences in these libraries (as well as in the linear peptide libraries described above) often consist of one or more degenerate positions, i.e., positions where two or more amino acids may be present, and one or more non-degenerate positions, i.e., positions where a single amino acid is present. Non-degenerate positions serve to orient the library to a binding site. Often 18 natural amino acids are present at a particular degenerate position (tryptophan and cysteine typically are not used because of analytical difficulties). The number of potential peptides in a library is Xⁿ, where X is the number of amino acids at a specific position and n is the number of degenerate positions.

The cyclic peptides typically have a sequence of between 5 and 25 amino acids, i.e., a 5-mer and a 25-mer. For example, a library of cyclic 8-mer peptides having n=6 and X=18 potentially will be composed of 34,012,224 peptides. In another example, a cyclic 10-mer peptide library where X=6 and n=6 may yield a library of 46,656 peptides. In a further example, a library consisting of cyclic 5-mer peptides having only three degenerate positions, i.e., n=3, with X=18, yields a library of 5,832 peptides. In practice, peptide libraries typically are synthesized by solid phase peptide synthesis using between about 0.1 mmole of resin and 0.5 mmole of resin. The yield of such a synthesis typically is about 20-100 mg of peptides having molecular weights between about 500 and 3000 Daltons.

With peptide libraries having high degeneracy, the preferred analytical method is Edman degradation. Accordingly, the natural or unnatural amino acids that can be used must be compatible with Edman degradation, i.e., α-amino acids. See, e.g., CELL (1993) 72:767-778; U.S. Pat. No. 5,532,167; and International Application No. PCT/US98/10876 (WO 98/54577). With libraries having low degeneracy, techniques other than Edman degradation can be used for analysis, e.g., liquid chromatography coupled with mass spectrometry (LC-MS), and any type of unnatural amino acid may be used, e.g., β-amino acids.

Cyclic peptide libraries typically are made as linear peptides by state-of-the-art solid phase peptide synthesis, allowing 18-20 natural amino acids to react simultaneously at a non-degenerate position and a single defined amino acid to react at a non-degenerate position of the linear peptide. The linear peptide then is cyclized, usually on the resin, or optionally after cleavage from the resin. Yields of the cyclization reaction typically are about 20-85%, so a substantial amount of linear peptide remains. Within a cyclic peptide library, linear peptide impurities or contaminants complicate the interpretation of analytical data. Accordingly, efficient evaluation of a cyclic peptide library and identification of cyclic peptides having desirable properties requires separation of the cyclic peptides from the linear peptide contaminants.

Since the linear peptides have molecular weights similar to the cyclic peptides, separation of cyclic peptides from linear peptide contaminants by techniques based on molecular weight, e.g., gel filtration, are ineffective. Moreover, separation of a cyclic peptide library from its linear peptide contaminants based on methods other than molecular weight is complicated further by the wide range of physical properties present in linear and cyclic peptides due to the variation in amino acid composition. That is, the cyclic and linear peptides are composed of many combinations of amino acids, and the individual peptides may be highly charged, either negatively or positively. This has led to the development of methods which use specific binding interactions to separate linear and cyclic peptides.

In International Application No. PCT/US98/10876, the cyclic peptide libraries are purified using a specific binding interaction between the linear peptides having a blocking agent, and a binding agent which interacts with the linear peptides but not with the cyclic peptides. More specifically, the blocking agent is biotin or an antigen, and the binding agent is avidin or streptavidin, or an antibody, respectively. The binding agents typically are immobilized on a column so that traditional affinity chromatography is used to effect the separation. However, affinity chromatography supports, e.g., avidin chromatography columns, typically are expensive with a limited useful lifetime. For example, an avidin column has a low capacity for biotinylated peptides compared to the number of moles of linear peptide which must be removed after a typical solid phase synthesis. Furthermore, regeneration of avidin columns is not easily accomplished.

Accordingly, given the large heterogeneity of a cyclic peptide library which often is in the presence of a highly diverse population of linear peptide contaminants, there is a need in the art for a simple, efficient, reliable, and cost effective method for purifying and analyzing cyclic peptide libraries prior to their further evaluation and use.

### SUMMARY OF THE INVENTION

It unexpectedly has been discovered that despite the heterogeneity of a cyclic peptide library and its linear peptide contaminants, the cyclic peptide library may be separated from the linear peptide contaminants using hydrophobic-hydrophobic interaction resulting in a more efficient, reliable, and economical way to purify cyclic peptide libraries. By associating a hydrophobic tag to the linear peptides, the chromatographic mobility of the linear peptides is altered sufficiently to overcome the diverse physical properties of the cyclic and linear peptides, e.g., peptide side chain properties, to permit their separation from the cyclic peptide library. Surprisingly, the attachment of a hydrophobic tag to the linear peptides provides sufficiently strong binding to a hydrophobic support to retain the linear peptides while the cyclic peptides readily are eluted.

More specifically, a cyclic peptide library may be purified by separating the cyclic peptide library from its linear peptide contaminants which have an attached hydrophobic tag, e.g., a fluorenyloxycarbonyl group (Fmoc), by contacting the cyclic and linear peptides with a hydrophobic support, e.g., a reversed phase chromatography column. As a result, the linear peptide contaminants are retained longer on the support and elute later than the cyclic peptide library, permitting the library of cyclic peptides to be collected substantially free from linear peptide contaminants. This technique is simple and cost effective as the fluorenyloxycarbonyl group is widely used as an N-terminal blocking group in peptide synthesis and many Fmoc-protected amino acids are commercially available. Furthermore, reversed phase high performance liquid chromatography (RP-HPLC) columns are widely used and procedures for regenerating such columns as well established.

Another aspect of the invention is methods for determining the proportion of linear peptide contaminant in a crude or purified library of cyclic peptides. Preferred methods of the invention exploit the distinction between the amino acid composition of the linear peptides and cyclic peptides, or the ability of linear peptides to be sequenced to the exclusion of cyclic peptides.

Another aspect of the invention is the compositions of matter which are produced by the methods of the invention. Since most state-of-the-art processes for separating non-cyclic peptide libraries involve attaching a separation tag to the peptides of interest, the compositions of matter which result from practicing methods of the invention provide novel compositions of matter. More specifically, subsequent to the association of the hydrophobic tag with the linear peptide contaminants after cyclization, the resulting compositions of matter typically are novel. Moreover, subsequent to separation of the cyclic peptide library from substantially all the linear peptide contaminants, the collected library typically is a unique composition of matter. Further screening and isolation of particular cyclic peptides from a library using methods of the invention also is encompassed by the present invention. That is, following isolation of a cyclic peptide library using methods of the invention, further isolation of a cyclic peptide having desired properties such as physiological activity is contemplated and included within the scope of the invention.

The foregoing, and other features and advantages of the invention, as well as the invention itself, will be more fully understood from the description, drawings, and claims which follow.

### DESCRIPTION OF THE DRAWINGS

FIGS. 1A-1G are mass spectra, HPLC chromatograms, and UV absorption spectra of the 8-mer cyclic peptide library (cyc[M1-X2-X3-X4-X5-X6-X7-N8]) of Example 1A. Figure 1A is a mass spectrum of the crude cyclic peptide library. FIG. 1B is a mass spectrum of the crude cyclic peptide library after treatment with piperidine. FIG. 1C is an HPLC chromatogram of the purification of the cyclic peptide library. FIG. 1D is an HPLC chromatogram of the purification of the cyclic peptide library after treatment with piperidine. FIG. 1E is UV absorption spectra of HPLC fractions of the cyclic peptide library which were collected at 30 min (trace labeled “A”) and at 50 min (trace labeled “B”). FIG. 1F is a mass spectrum of the HPLC fractions collected between 14-35 min. FIG. 1G is a mass spectrum of the fractions collected between 35-60 min.

FIGS. 2A-2D are mass spectra and HPLC chromatograms of the 8-mer cyclic peptide library (cyc[M1-X2-X3-X4-X5-X6-X7-N8]) of Example 1B. FIG. 2A is a mass spectrum of the crude cyclic peptide library. FIG. 2B is an HPLC chromatogram of the purification of the cyclic peptide library. FIG. 2C is a mass spectrum of the HPLC fractions collected between 14-35 min. FIG. 2D is a mass spectrum of the HPLC fractions collected between 48-60 min.

FIGS. 3A-3D are mass spectra and HPLC chromatograms of the 8-mer cyclic peptide library (cyc[M1-X2-X3-X4-X5-X6-X7-N8]) of Example 1C. FIG. 3A is a mass spectrum of the crude cyclic peptide library. FIG. 3B is an HPLC chromatogram of the purification of the cyclic peptide library. FIG. 3C is a mass spectrum of the HPLC fractions collected between 14-35 min. FIG. 3D is a mass spectrum of the HPLC fractions collected between 48-60 min.

FIGS. 4A-4D are mass spectra and HPLC chromatograms of the 6-mer cyclic peptide library (cyc[M1-X2-X3-X4-X5-N6]) of Example 1. FIG. 4A is a mass spectrum of the crude cyclic peptide library. FIG. 4B is an HPLC chromatogram of the purification of the cyclic peptide library. FIG. 4C is a mass spectrum of the HPLC fractions collected between 14-35 min. FIG. 4D is a mass spectrum of the HPLC fractions collected between 48-60 min.

FIG. 5 is a bar graph showing the percentages of linear peptide contaminants versus cyclic peptides in crude cyclic peptide libraries (a 6-mer, 7-mer, 8-mer, 10-mer, and 12-mer) prior to purification. The percentages were determined using a preferred method of the invention involving amino acid analysis of a norvaline residue coupled to the linear peptide contaminants subsequent to cyclization.

### DETAILED DESCRIPTION OF THE INVENTION

It unexpectedly has been discovered that despite the heterogeneity of a cyclic peptide library and its linear peptide contaminants, i.e., the uncyclized linear peptides sequences, the cyclic peptide library may be separated from linear peptide contaminants using hydrophobic-hydrophobic interaction. The methods of the invention result in a simple, efficient, reliable, and economical way to purify cyclic peptide libraries. More specifically, a cyclic peptide library may be purified by separating the cyclic peptides from linear peptide contaminants which have an associated hydrophobic tag by contacting the cyclic and linear peptides with a hydrophobic support. As a result, linear peptide contaminants are retained longer on the support and elute later than the cyclic peptide library, permitting the library of cyclic peptides to be collected substantially free from linear peptide contaminants.

As used herein, “library of cyclic peptides” or “cyclic peptide library” is understood to mean two or more cyclic peptides where the two or more cyclic peptides within the library are composed of different sequences. Of course, different amounts of each cyclic peptide may be present within the library. Often a cyclic peptide library may contain fifty or more cyclic peptides. Preferably, the cyclic peptide library contains one hundred or more, or five hundred or more cyclic peptides. More preferably, a cyclic peptide library may contain one thousand or more, or five thousand or more cyclic peptides. In certain embodiments, a cyclic peptide library may contain ten thousand or more cyclic peptides.

As used herein, “hydrophobic-hydrophobic” is understood to mean an interaction in which is the driving force is thermodynamic minimization of the hydrophobic contact area between hydrophobic regions, domains, or entities, and the relatively polar solvent medium used in the separation of cyclic peptides from linear peptide contaminants. The hydrophobic regions, domains, or entities themselves typically are sufficiently hydrophobic to be non-water wettable and water insoluble.

As used herein, “hydrophobic tag” is understood to mean a chemical group, substituent, or entity which may be associated with a linear peptide to provide a means to effect the separation of linear peptides from cyclic peptides using hydrophobic-hydrophobic interaction. That is, a hydrophobic tag preferably associates with other hydrophobic materials or compositions which typically are composed primarily of carbon and hydrogen.

As used herein, “hydrophobic support” is understood to mean a composition of matter that has interactive regions associated with it which permit hydrophobic-hydrophobic interactions to occur. Support materials include, but are not limited to, chromatography matrices, resins, filters, and membranes. Reversed phase chromatography media are preferred, and more particularly, C18 reversed phase chromatography media.

As used herein, the term “natural amino acid” is understood to mean any one of the twenty amino acids defined in the genetic code. See, e.g., Stryer, L. (1988) “Biochemistry,” 3^{rd} edition, W. H. Freeman and Company, New York.

As used herein, “non-natural amino acid” is understood to mean any amino acid which is not defined in the genetic code. A non-natural amino acid may be a naturally occurring amino acid such as β-alanine. Non-natural amino acids include, but are not limited to, α-amino acids, β-amino acids, γ-amino acids, halogenated amino acids, phosphorylated amino acids, and amino acids of any optical configuration.

As used herein, “substantially pure cyclic peptide library” is understood to mean a cyclic peptide library which contains less than about 5% of linear peptide contaminants. Preferably, the cyclic peptide library contains less than about 2% of linear peptide contaminants. Accordingly, the proportion of linear peptide contaminants in a substantially pure cyclic peptide library typically is less than about 1:20, and preferably less than 1:50.

Generally, synthesis of a cyclic peptide library is accomplished by techniques known to those skilled in the art, usually derived from procedures developed for the synthesis of single cyclic peptides and often using an automated synthesizer. See, e.g., International Application No. PCT/US94/07687 (WO 95/01800); International Application No. PCT/US98/10876 (WO 98/54577); Blackburn et al. (1997) METHODS ENZYMOL. 289:175-98; Wiesmuller, K. -H. et al., “Peptide and Cyclopeptide Libraries: Automated Synthesis, Analysis and Receptor Binding Assays,” in Jung, G. (ed.) (1996) “Peptide and Nonpeptide Libraries—A Handbook,” VCH, Weinheim, Germany; and references cited therein.

Cyclic peptide libraries often are synthesized by one of two general strategies both of which involve first synthesizing linear peptides using automated solid phase peptide methods. Thereafter, cyclization can be performed on the resin, followed by deprotection and cleavage. Alternatively, after synthesis of the linear peptides, the protected peptides are cleaved from the resin, followed by cyclization and deprotection. With on-resin or off-resin peptide cyclization strategies, the association of a hydrophobic tag with the linear peptides occurs after cyclization and before deprotection of amino acid side chains. It should be noted that in addition to the full length linear peptides and their counterpart cyclized products, truncated linear and cyclic peptides may be present which typically are not resolved from the intended full length peptides.

Regardless of the technique or method used to synthesize the cyclic peptide library, linear peptide contaminants typically remain after cyclization and subsequent deprotection/deblockage and cleavage, if necessary. Accordingly, prior to deprotection and optional cleavage, a hydrophobic tag is associated with the linear peptides. That is, the association step may occur with the cyclic peptides and linear peptides free in solution. However, linear peptides typically are synthesized attached to a solid support or resin so that the association of the hydrophobic tag typically occurs while the peptides are attached to the solid support or resin.

For example, as known in the art, a number of linear peptides may be synthesized by adding successive amino acid residues to an amino acid attached to a solid support, optionally through a linker. After the desired number of amino acid residues are added, two or more linear peptides may be cyclized to form a library of two or more cyclized peptides. Since yields of the cyclization step are less than perfect, at least one linear peptide does not cyclize creating a linear peptide contaminant. Prior to deprotection/deblockage and release of the cyclic peptides and linear peptide contaminant from the solid support, a hydrophobic tag is associated, e.g., by coupling using covalent bonding, with the linear peptide contaminant thereby providing the means or “handle” with which to separate the linear peptide from the cyclic peptides. The peptides then are deprotected/deblocked and cleaved from the solid support, e.g., using a acid such as trifluoroacetic acid, and the resulting library of cyclic peptides and the linear peptide having the hydrophobic tag is contacted with a hydrophobic support to effect the separation. Finally, the cyclic peptide library which is substantially free of linear peptide contaminants is collected from the hydrophobic support.

Hydrophobic tags useful in the practice of the invention include, but are not limited to, a fluorenylmethoxycarbonyl group (Fmoc); appropriately derivatized Fmoc groups (see, e.g., Ball et al., “Selective purification of large synthetic peptides using removeable chromatographic probes,” in Giralt, E. et al. (eds.) (1990) PEPTIDES 1990, PROCEEDINGS OF THE TWENTY-FIRST EUROPEAN PEPTIDE SYMPOSIUM, SEPTEMBER 2-8, 1990, PLATJA D'ORO, SPAIN, PAGES 323-325; Ball et al. (1992) INT. J. PEPTIDE PROTEIN RES. 40: 370-379; and Ramage et al. “Methodology for Chemical Synthesis of Proteins,” in Epton, R. (ed.) (1996) INNOVATION AND PERSPECTIVES IN SOLID PHASE SYNTHESIS & COMBINATORIAL LIBRARIES, COLLECTED PAPERS FROM THE FOURTH INTERNATIONAL SYMPOSIUM, SEPTEMBER 12-16, 1995, EDINBURGH, SCOTLAND, UK, PAGES 1-10); and n-alkyl groups (see, e.g., Garcia-Echeverria (1995) J. CHEM. SOC., CHEM. COMMUN. 779-780).

Many techniques of associating hydrophobic tags with linear peptides are known. A preferred technique is coupling at least one natural or non-natural amino acid to the α-nitrogen atom at the N-terminal end of a linear peptide (see Example 1). Often only one natural or non-natural amino acid is coupled to the linear peptide. In this case, the natural or non-natural amino acid has a hydrophobic tag associated with it (see, e.g., Examples 1B-1D). However, more than one natural or non-natural amino acid may be coupled to the linear peptide prior to association of the hydrophobic tag with the linear peptide (see, e.g., Example 1A). In this particular case, the last coupled amino acid has an associated hydrophobic tag thereby permitting purification of the cyclic peptide library. Preferred natural and non-natural amino acids having a hydrophobic tag useful in coupling to the linear peptide include, but are not limited to, N-(9-fluorenylmethoxycarbonyl)alanine (Fmoc-Ala), N-(9-fluorenylmethoxycarbonyl)norvaline (Fmoc-Nva), N-(9-fluorenylmethoxycarbonyl)leucine (Fmoc-Leu), and N-(9-fluorenylmethoxycarbonyl)β-alanine (Fmoc-βAla).

Another preferred technique is directly coupling the hydrophobic tag to the α-nitrogen atom at the free N-terminal end of the linear peptide thereby capping the linear peptide sequence (see Example 2). Useful reagents for direct coupling include, but are not limited to, N-(9-fluorenylmethoxycarbonyloxy)succinimide (Fmoc-OSu), and 9-fluorenylmethyl chloroformate (Fmoc-Cl). Using a preferred hydrophobic tag as an example, after cyclization, a fluorenylmethoxycarbonyl (Fmoc) group may be directly coupled to the uncyclized linear peptides under the appropriate reaction conditions to form a linear peptide having an associated hydrophobic tag, i.e., an Fmoc group. Subsequently, if a solid support is used in the synthesis, the linear peptide having a fluorenylmethoxycarbonyl group can be separated from the cyclic peptides following cleavage of the peptides from the solid support.

As described elsewhere, following association of a hydrophobic tag with a linear peptide, the library of cyclic peptides and the linear peptide having an associated hydrophobic tag are separated using a hydrophobic support. Preferably the separation is effected by introducing the mixture of cyclic and linear peptides to a chromatography column which is packed with a medium having hydrophobic interaction regions. The preferred chromatographic technique is reversed phase high performance liquid chromatography (RP-HPLC). An eluting mobile phase then is passed through the column under the appropriate conditions and parameters to effect an efficient separation which resolves the cyclic peptides and permits collection of the cyclic peptide library which elutes first (see Example 1). Other supports having hydrophobic interactive regions such as resins, filters and membranes also may be used as known to those skilled in the art.

Another aspect of the invention is methods for determining the proportion of linear peptide contaminants in a crude or purified library of cyclic peptides. Typically, the proportion is determined subsequent to the purification of the cyclic peptide library to indicate the purity of the cyclic peptide library prior to further experimentation with or screening of the library, and/or isolation of cyclic peptides having desirable properties. Preferred methods for determining this proportion rely on the distinction between the amino acid composition and/or other properties of the linear and cyclic peptides.

More specifically, the difference in amino acid composition of the linear peptides and the cyclic peptides may be exploited to determine the amount of each thereby permitting calculation of the proportion. For example, determining the amount of a natural or non-natural amino acid which is present only in the linear peptides, and determining the amount of a natural or non-natural amino acid which is common to both the linear and cyclic peptides will permit the calculation of the proportion of linear peptide contaminants in a cyclic peptide library (see Example 3).

That is, in the practice of this method of the invention, an amino acid which is not present in the cyclic peptide library is added to the linear peptide contaminant after cyclization. Determination of the moles of the amino acid present only in the linear peptide then indicates the moles of linear peptide contaminants present. The moles of amino acid typically are determined using standard amino acid analysis techniques known in the art. In addition, the moles of an amino acid which is common to both the linear and cyclic peptides, e.g., a C-terminal asparagine, provides the total amount of both cyclic and linear peptides present. Subsequently, comparison of the moles of the two different amino acids permits the calculation of the proportion or percentage of linear peptide contaminants in the crude or purified cyclic peptide library.

Particularly useful in the practice of this technique is to couple a non-natural amino acid having a hydrophobic tag, e.g., Fmoc-norvaline, to the linear peptide so that the detection and quantification of the linear peptides is simplified since most cyclic peptide libraries of interest are composed of naturally occurring amino acids. It should be noted that if the amino acid common to both linear and cyclic peptides may be present in degenerate positions, as well as a non-degenerate position, a correction needs to be made to account for the multiple occurrences of the common amino acid to provide a meaningful proportion.

Another technique for determining the proportion exploits the capability of a linear peptide to be degraded by enzymatic processes to which cyclic peptides are resistant. In this method, the total amount of peptide typically is determined in a sample collected after separation of the linear and cyclic peptides. Subsequently, a measured aliquot of the sample is subjected to one of a variety of sequencing techniques such as Edman degradation then resequenced often using an automated peptide synthesizer. The amount of linear peptide present in the aliquot can be determined by measuring the amount of peptide sequenced, often corrected by a standard conversion factor. As a result, the proportion of linear peptide contaminants in the cyclic peptide library may be calculated.

Another aspect of the invention is the compositions of matter which are produced by methods of the invention. Since most state-of-the-art processes for separating peptides involve attaching a separation tag to the desired peptides, compositions of matter which result from practicing the methods of the invention may be novel. More specifically, in the practice of methods of the invention, subsequent to the association of the hydrophobic tag with the linear peptides, opposed to prior methods, the resulting composition of matter typically is unique. Moreover, subsequent to separation of the cyclic peptide library from substantially all the linear peptide contaminants using methods of the invention, the collected library often will be a composition of interest.

Further screening and isolation of particular cyclic peptides following practice of methods of the invention also is encompassed by the present invention. That is, following isolation of a cyclic peptide library using methods of the invention, further isolation of a cyclic peptide having desired properties such as physiological activity is contemplated and included within the scope of the invention.

For example, the cyclic peptide libraries purified by methods of the invention can be used to deduce the characteristics of the active sites of a variety of proteins. Individual peptides of a cyclic peptide library may be screened through a chromatography column having immobilized binding sites and the properties of the tightly bound peptides can provide characteristics of the binding site of the target protein. See, e.g., Songyang, Z. et al. (1994) MOL. CELL. BIOL. 14: 2777-2785. Cyclic peptide libraries also can be subjected to the activity of proteases, which will hydrolyze some components of a cyclic peptide library, and make those peptides susceptible to Edman degradation (see, e.g., WO 98/54577, Example 3). In this way the selectivity of the protease site for cyclic peptide substrates can be obtained. In another example, the selected components of a cyclic peptide library can be phosphorylated and the characteristics of protein kinase binding sites deduced (see, e.g., WO 98/54577, Example 4). Knowledge of the characteristics of a binding site then can allow the design of high affinity non-peptide inhibitors of the target protein. In addition, the individual cyclic peptides of a cyclic peptide library may have useful pharmaceutical properties or be used to develop other related cyclic peptides which bind more selectively or with higher affinity. These individually identified cyclic peptides (or a combination thereof) can become drug candidates, e.g., the subject of an investigational new drug application (INDA) and/or a new drug application (NDA).

The invention is illustrated further by the following non-limiting examples.

### EXAMPLES

### Example 1

### Purification of Cyclic Peptide Library by Coupling at Least One Amino Acid Having a Hydrophobic Tag

### Example 1A

The 8-mer cyclic peptide library cyc[M1-X2-X3-X4-X5-X6-X7-N8] was synthesized. X denotes a degenerate position containing 19 natural amino acids (no cytsteine), and M and N stand for methionine (Met) and asparagine (Asn), respectively.

### Library Synthesis

Cyclic peptides libraries were synthesized on an ABI 433A peptide synthesizer with 9-fluorenylmethoxycarbonyl (Fmoc) protecting groups using a Rink Amide Methylbenzhydrylamine (MBHA) resin (0.5 g, 0.54 mmol/g, 0.27 mmol). The C-terminal residue, FmocAsp(ODmab) where Dmab is 4-{N-[1-(4,4-dimethyl-2,6-dioxocyclohexylidene)-3-methylbutyl]amino}benzyl ester, is first attached through the side chain at a four fold molar excess over the moles of resin. Linear peptide synthesis is accomplished with O-benzotriazol-1-yl)-N,N′,N″,N′″-tetramethyluronium/1-hydroxybenzotriazole/diisopropylamine (HBTU/HOBT/DIEA) (1 equivalent per peptide resin). Treatment of the resin with 2% hydrazine (400 seconds) removes the ODmab and Fmoc groups, providing a free N-terminal amino group and free C-terminal carboxyl group of the peptide. (The peptide is attached to the resin through the aspartic acid (Asp) side chain.) Head to tail cyclization is accomplished with O-(7-azabenzotriazol-1-yl)-N,N′,N″,N′″-tetramethyluronium/diisopropylamine (HATU/DIEA) (1 equivalent per peptide resin), repeated four times.

Specifically in Example 1A, after the cyclization step, the uncyclized peptides were coupled with a cysteine residue (Cys), followed by capping with N-(9-fluorenylmethoxycarbonyl)norvaline (Fmoc-Nva) using the standard peptide synthesis protocol as described above. As a result, the full length linear peptides had the sequence [Fmoc-Nva-Cys-M1-X2-X3-X4-X5-X6-X7-N8].

The cyclic peptide library and Fmoc-tagged linear peptides concurrently were cleaved from the resin and the amino acid side chains deprotected with trifluoroacetic acid/phenol/thioanisole/1,2-ethanedithiol/water (82.5%/5%/5%/2.5%/5%). The peptide mixture then was precipitated from methyl tert-butyl ether (MTBE), washed with MTBE, re-suspended in 10% acetic acid, and freeze dried (lyphilized).

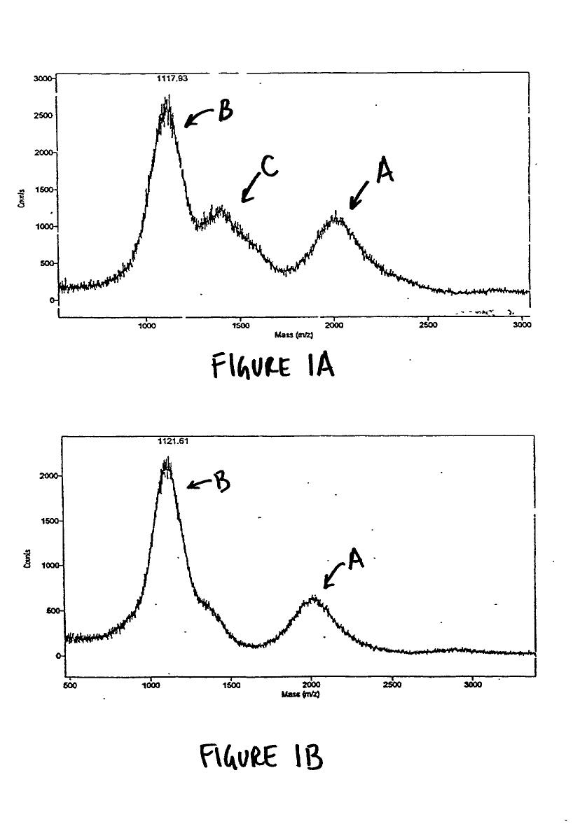

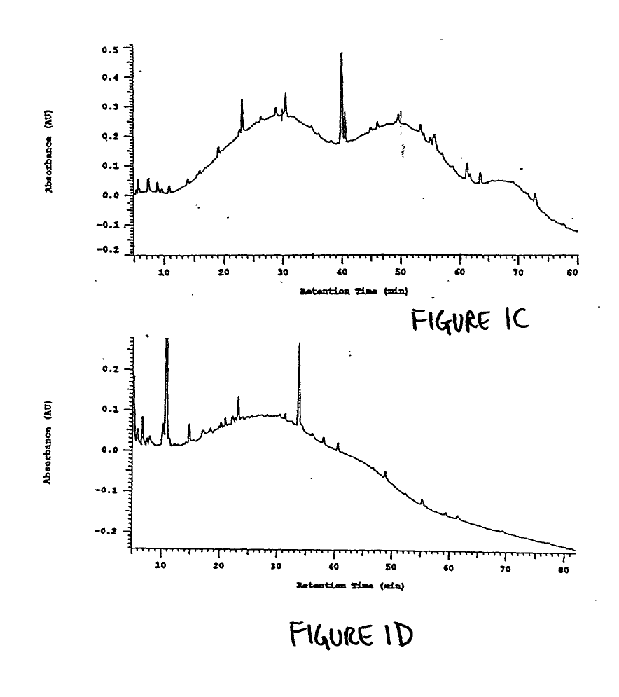

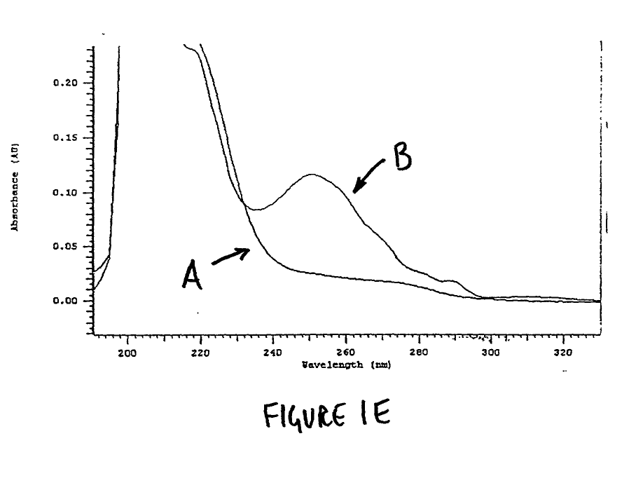

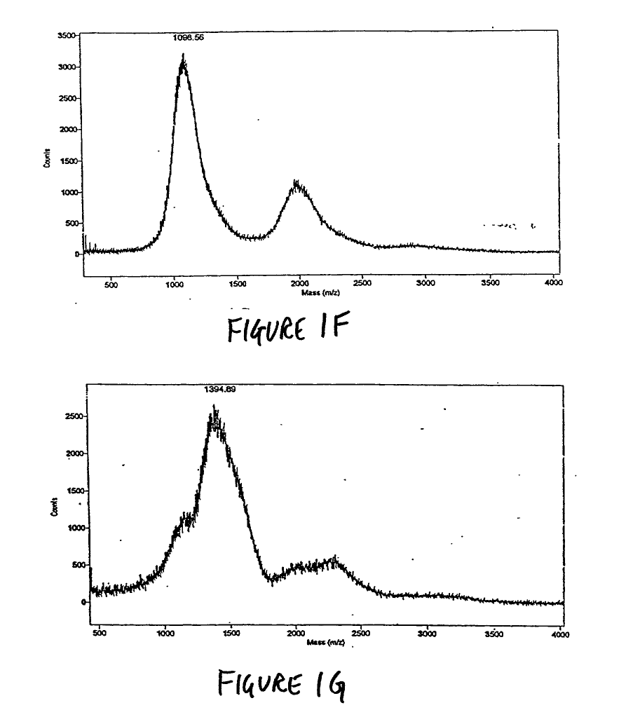

The mass spectrum of the crude peptide mixture (FIG. 1A) shows a small peak at a molecular weight (MW) of about 2000 (peak labeled as “A”) and a large peak at a MW of about 1100 (peak labeled as “B”) with a large hump or shoulder (peak labeled as “C”). As shown in FIG. 1B, the hump diminished after the crude peptide mixture was treated with 50% piperidine to remove the Fmoc group.

### Library Purification

A Hitachi D-7000 HPLC system with a diode array detector was used with a C18 reversed phase column (10 μm, 10 mm×250 mm, 300 Å pore, Vydac 238TP1010 from The Nest Group, Southborough, Mass.). Reagents used were trifluoroacetic acid (TFA), methyl tert-butyl ether (MTBE), HPLC water, acetonitrile (CH₃CN), and isopropanol (IPA). Buffer A was 0.1% TFA, and buffer B was 10% buffer A, 70% CH₃CN, and 20% IPA. The flow rate was 2.5 mL/min.

Ultraviolet (UV) absorption was monitored at the wavelength range of 200 nm to 320 nm and the absorption at 214 nm was recorded as a fraction of time. Appropriate fractions were collected, reduced in volume, and lyophilized overnight. The different fractions were characterized by matrix assisted laser desorption ionization time of flight mass spectrometry (MALDI-TOF MS) at Louisiana State University Core Laboratory (New Orleans, La.).

In Example 1A, the crude peptide library and Fmoc-tagged linear peptide contaminants, both before and after piperidine treatment, were subjected to reversed phase HPLC. The linear gradient was 5% buffer B (5%B) to 70% buffer B (70%B) in 60 min. FIGS. 1C (before piperidine treatment) and 1D (after piperidine treatment) are the HPLC chromatograms during purification. In FIG. 1C, the chromatogram shows a peak at about 30 min representing the cyclic peptide library, a peak at about 50 min representing the Fmoc-tagged linear peptide contaminants, and a peak at about 67 min which was not characterized. In Figure 1D, the chromatogram of the crude peptide library which was treated with piperidine to remove the Fmoc groups from the peptides shows a major peak at about 30 min with the near disappearance of the peak at about 50 min.

As shown in Figure 1E, the UV absorption spectrum of the HPLC fraction of the crude peptide library (before piperidine treatment) at 30 min. (trace labeled “A”) is characterized by a single peak with a maximum between 205 nm and 220 nm due to peptide bonds, indicating that the cyclic peptides were eluted earlier than the Fmoc-labeled linear peptide contaminants. Subsequently, the HPLC fraction analyzed at 50 min (trace labeled “B”) contained the linear peptide contaminants having an associated Fmoc group with its characteristic UV absorption between 240 nm to 300 mn.

These results were further evidenced by mass spectrometry. In FIG. 1F, the mass spectrum of the fractions collected between 14-35 min demonstrates that the peptides having an associated Fmoc group were removed completely as shown by the lack of a peak near a MW of about 1400. The major peak at a MW of about 1100 in FIG. 1F represents pure cyclic peptides, with the minor peak representing cyclic peptide dimers. As seen in FIG. 1G, the mass spectrum of the fractions collected between 35-60 min produce a large peak at a MW of about 1400 indicating Fmoc-tagged linear peptide contaminants.

### Example 1B

An 8-mer cyclic peptide library cyc[M1-X2-X3-X4-X5-X6-X7-N8] was synthesized as in Example 1A. However, after the cyclization step, the uncyclized peptides were capped with N-(9-fluorenylmethoxycarbonyl)alanine (Fmoc-Ala) using standard peptide synthesis protocol as described above. As a result, the full length linear peptides had the sequence [Fmoc-Ala-M1-X2-X3-X4-X5-X6-X7-N8]. The cyclic peptide library and linear peptides then were cleaved and deprotected as described in Example 1A.

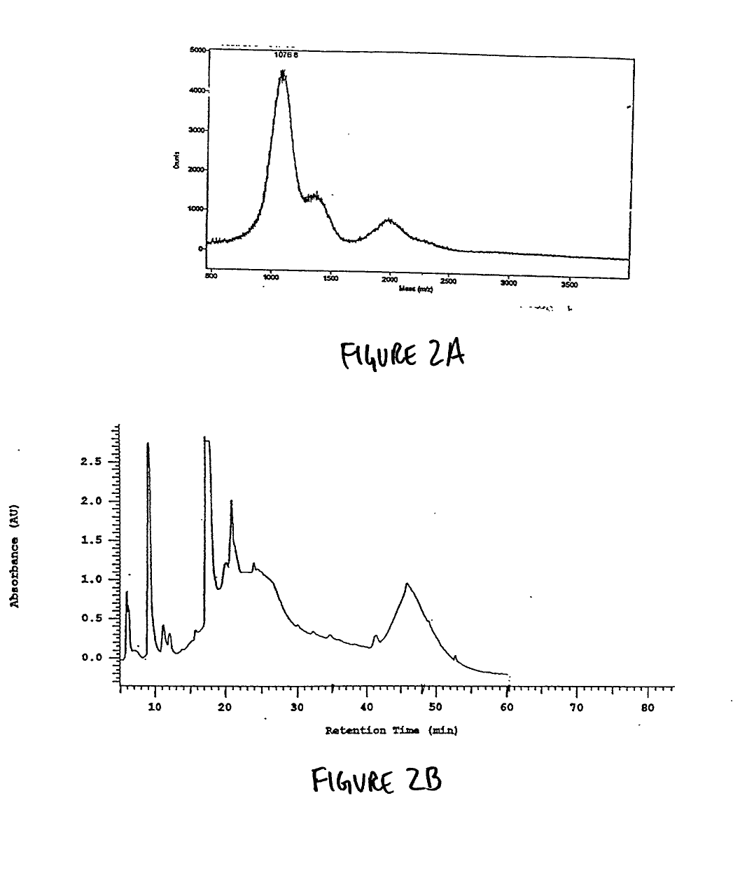

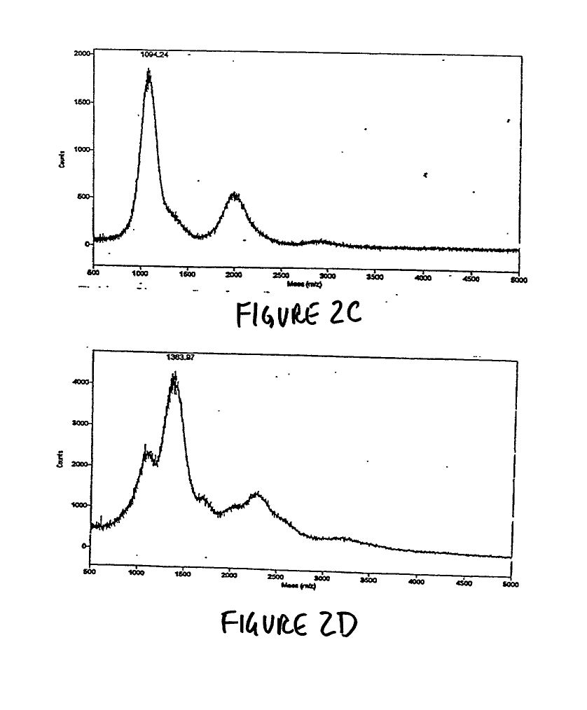

As shown in FIG. 2A, the mass spectrum of the crude peptide mixture shows one major peak at a MW of about 1100, representing the cyclic peptide library. The shoulder peak at a MW of about 1400 represents the Fmoc-capped linear peptide contaminants and the minor peak at a MW of about 2000 represents cyclic peptide dimers.

Purification and analysis of the crude peptide mixture was conducted as described in Example 1A, however, the HPLC gradient was modified to 5%B to 35%B for 15 minutes, then maintained at 35% B for 20 minutes, followed by 35%B to 95%B for 20 minutes. As shown in FIG. 2B, the HPLC chromatogram generally shows two bands of peptide distributions. Accordingly, fractions were collected at about 14-35 min (yield 25.0 mg), and 48-60 min (yield 3.0 mg). The earlier eluting band between 14-35 min contained only cyclic peptides showing strong UV absorption only between about 190 nm to 230 nm. The mass spectrum of this fraction (FIG. 2C) showed relatively no shoulder to the peak at a MW of about 1100. The mass spectrum of the later eluting band between about 48-60 min (FIG. 2D) indicated that this fraction contained mainly peptides having an associated Fmoc group as demonstrated by the large peak at a MW of about 1400.

### Example 1C

Another cyclic peptide library was synthesized as in Example 1B although under low loading conditions, i.e., a reduced density of peptides on the resin, and X was not cysteine or methionine. After the cyclization step, the uncyclized peptides were capped with N-(9-fluorenylmethoxycarbonyl)norvaline (Fmoc-Nva) using standard peptide synthesis protocol as described in Example 1A. As a result, the full length linear peptides had the sequence [Fmoc-Nva-M1-X2-X3-X4-X5-X6-X7-N8]. The cyclic peptide library and linear peptides then were cleaved and deprotected as described in Example 1A.

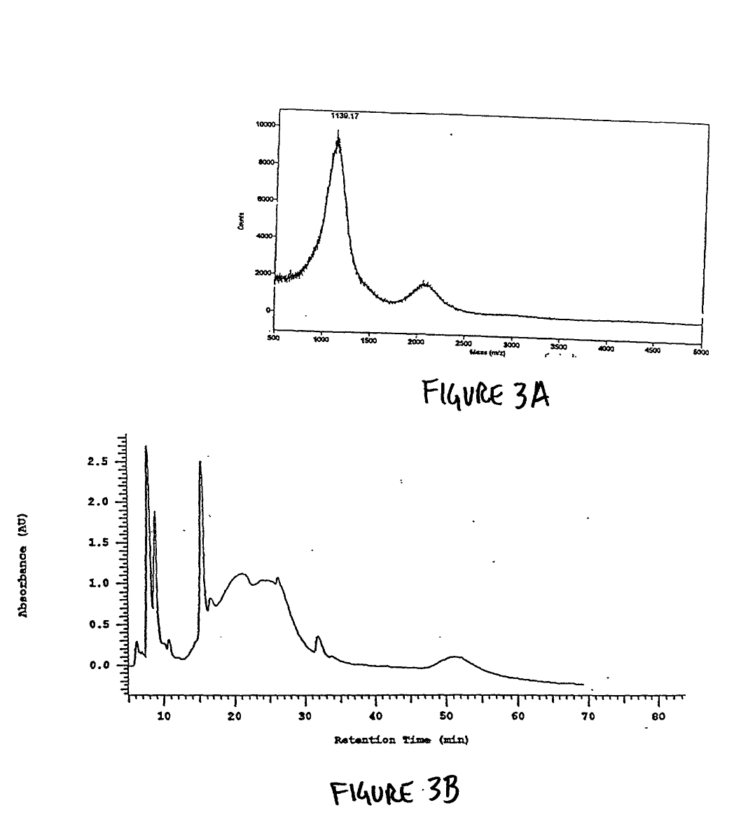

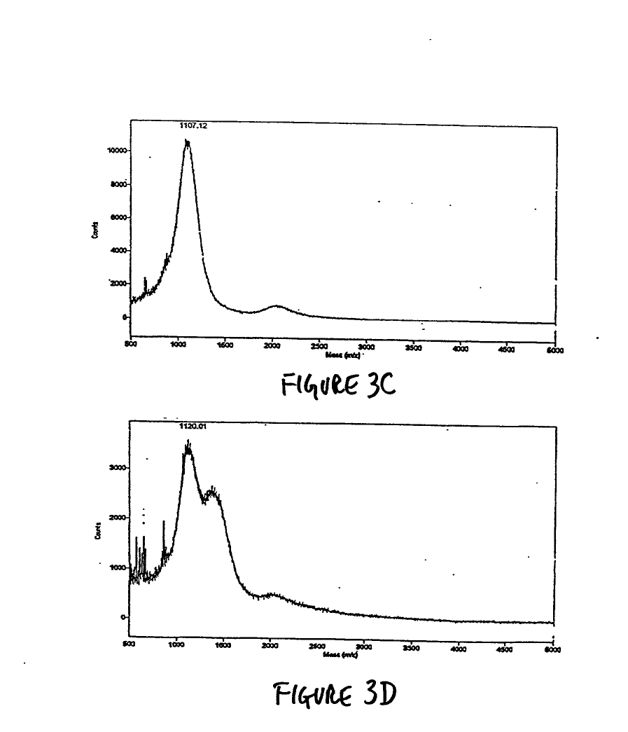

Purification and analysis was conducted as in Example 1B. The mass spectrum (FIG. 3A) of the crude peptide mixture showed little or no amounts of Fmoc-tagged linear peptide contaminants. Following purification (the HPLC chromatogram is shown in FIG. 3B), the mass spectrum of the earlier eluting fractions between about 14-35 min (yield 12.4 mg) (FIG. 3C) demonstrated that the cyclic peptide library was substantially pure (peak at a MW of about 1140) with a small amount of cyclic peptide dimers present (peak at a MW of about 2200). The mass spectrum of the later eluting fractions between about 48-60 min (yield 2.0 mg) which contain Fmoc-tagged linear peptide contaminants is shown in FIG. 3D.

### Example 1D

The 6-mer cyclic peptide library cyc[M1-X2-X3-X4-X5-N6] was synthesized, capped, and cleaved and deprotected as in Example 1A, although X was not cysteine or methionine. The resulting sequence of the full length linear peptides was [Fmoc-Ala-M1-X2-X3-X4-X5-N6].

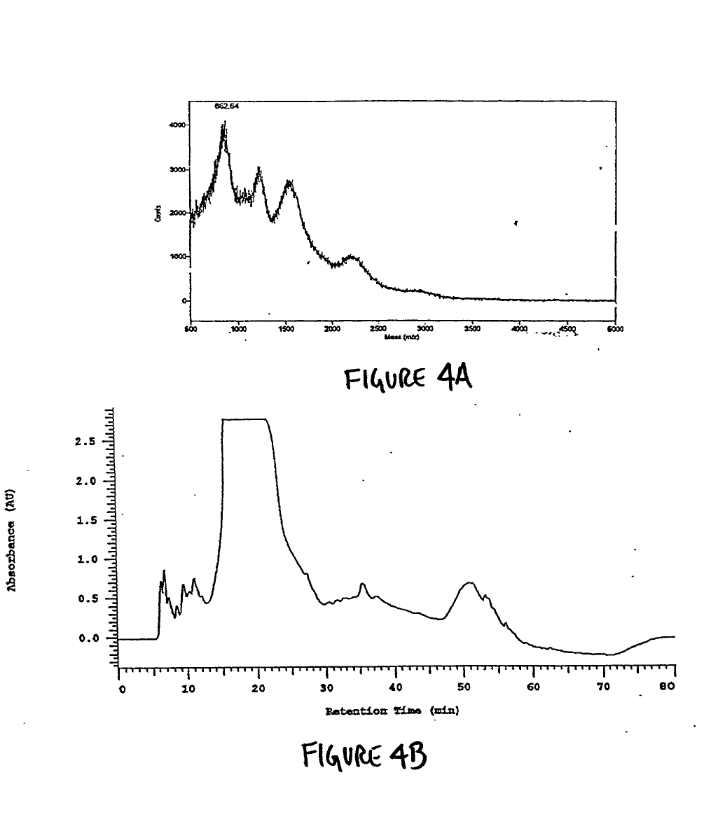

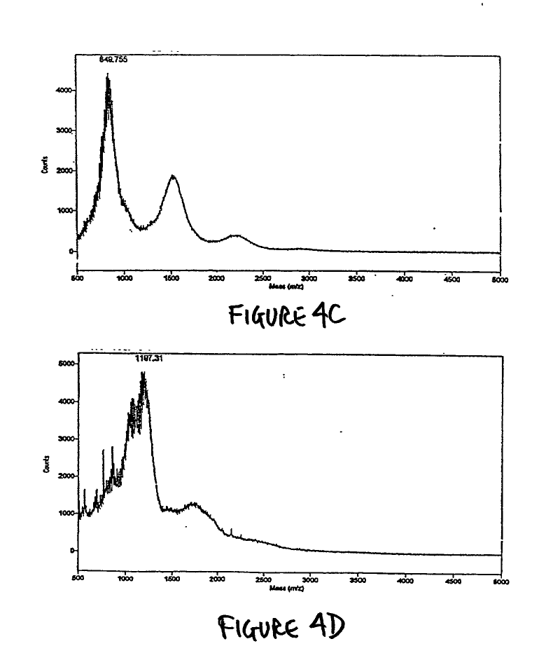

Purification and analysis of the crude peptide mixture was conducted as in Example 1B. The mass spectrum (FIG. 4A) of the crude peptide mixture showed large amounts of impurities as also seen in the HPLC chromatogram (FIG. 4B). However, following purification, the mass spectrum of the earlier eluting fractions between about 14-35 min (yield 23.6 mg) (FIG. 4C) indicated that the cyclic peptide library (peak at a MW of about 850) was substantially free of Fmoc-tagged linear peptide contaminants and other impurities, although a small amount of cyclic peptide dimers (peak at a MW of about 1550) and cyclic peptide trimers (peak at a MW of about 2300) were present. The mass spectrum of the later eluting fractions between about 48-60 min (yield 2.2 mg) which contains the Fmoc-tagged linear peptide contaminants (peak at a MW of about 1200) is shown in FIG. 4D.

### Example 2

### Purification of Cyclic Peptide Library By Direct Coupling of a Hydrophobic Tag

Following synthesis of a cyclic peptide library generally following the method of Example 1, except no additional residues were added after cyclization, the resin (0.5 g, 0.54 mmol/g, 0.27 mmol) was transferred to a 10 mL syringe containing a polypropylene frit. The resin was washed with dimethylformamide (DMF) (10 mL) and methylene chloride (CH₂Cl₂) (10 mL). Subsequently, the following were added in the indicated order to the washed resin: N-(9-fluorenylmethoxycarbonyloxy)succinimide (Fmoc-OSu) (5 equiv, 455 mg, 1.35 mmol), DMF (4 mL), and diisopropylethylamine (DIEA) (5 equiv, 235 μL). The reaction was shaken for 1 h, then washed with DMF (10 mL) and CH₂Cl₂ (10 mL), and dried in vacuo for 18 h to insure dryness.

Subsequent to direct coupling of the hydrophobic tag to the linear peptides, the peptides are cleaved from the resin, deprotected, purified, and analyzed as described above in Example 1.

### Example 3

### Determining Proportion of Linear Peptide in the Purified Cyclic Peptide Library Using Ratio of Amino Acids

The following series of cyclic peptide libraries were made generally following the method of Example 1.

| | |
|---|---|
| 6-mer | cyc[X1-X2-X3-X4-X5-N6] |
| 7-mer | cyc[X1-X2-X3-X4-X5-X6-X7-N7] |
| 8-mer | cyc[X1-X2-X3-X4-X5-X6-X7-N8] |
| 10-mer | cyc[X1-X2-X3-X4-X5-X6-X7-X8-X9-N10] |
| 12-mer | cyc[X1-X2-X3-X4-X5-X6-X7-X8-X9-X10-X11-N12] |

After cyclization, Fmoc-norvaline (Fmoc-Nva) was attached to the linear peptides using standard peptide synthesis protocol as described above. Subsequently, the peptides generally were cleaved and deprotected as described in Example 1. The unpurified peptide libraries then were submitted for amino acid analysis to determine the mole fraction of norvaline in each library. Amino acid analysis was conducted by Brigham and Women's Hospital Biopolymer Laboratory, Boston, Mass.

Using the results from the amino acid analysis, the calculation of the proportion of linear peptide contaminants in a cyclic peptide library, or any other common relationship between the amount of linear peptide contaminants in the cyclic peptide library, readily is accomplished. For example, the percentage of linear peptide contaminants was calculated using the formula:

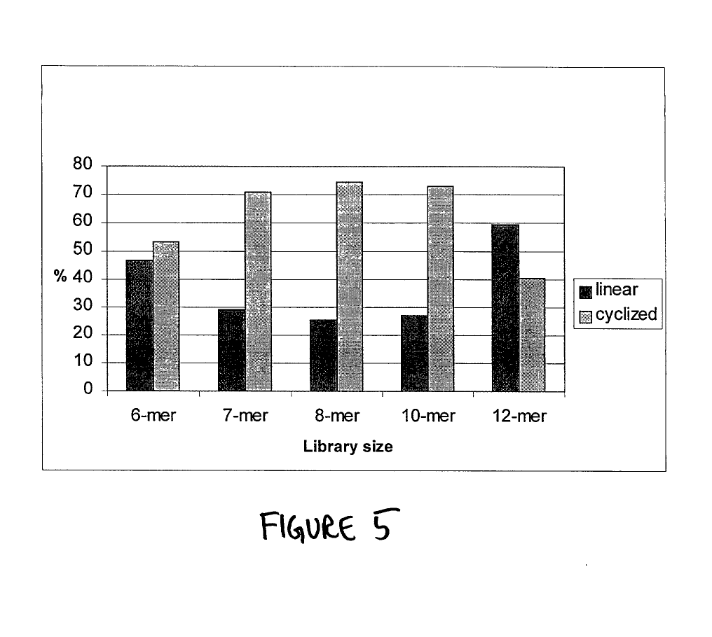

% linear peptide contaminant=100×[N×R/(1-R)]

where N is the ring size, and R is the mole fraction of norvaline. The results of this calculation for each of the crude cyclic peptide libraries in this Example are shown in FIG. 5.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are intended to be embraced therein.

Each of the patent documents and scientific publications disclosed hereinabove is incorporated by reference herein.

What is claimed is:

1. A method of separating a cyclic peptide library from linear peptide contaminants comprising the steps of:
(a) providing a library of two or more cyclic peptides, and a linear peptide comprising a hydrophobic tag;
(b) separating the library of two or more cyclic peptides from the linear peptide using a hydrophobic support; and
(c) collecting the library of two or more cyclic peptides from the hydrophobic support.

2. The method of claim 1 wherein the step of providing comprises the steps of:
(a′) cyclizing two or more linear peptides to form a library of two or more cyclic peptides where at least one linear peptide did not cyclize; and
(a″) associating a hydrophobic tag to the at least one linear peptide which did not cyclize.

3. The method of claim 2 wherein associating the hydrophobic tag comprises coupling at least one natural or non-natural amino acid to the linear peptide where the last coupled natural or non-natural amino acid comprises the hydrophobic tag.

4. The method of claim 3 wherein the last coupled natural or non-natural amino acid is N-(9-fluorenylmethoxycarbonyl)norvaline or N-(9-fluorenylmethoxycarbonyl)alanine.

5. The method of claim 2 wherein associating the hydrophobic tag comprises directly coupling the hydrophobic tag to the linear peptide.

6. The method of claim 5 wherein directly coupling the hydrophobic tag uses N-(9-fluorenylmethoxycarbonyloxy)succinimide.

7. The method of claim 1 further comprising the steps of:
(d) determining the proportion of linear peptide in the library of two or more cyclic peptides.

8. The method of claim 7 wherein the step of determining the proportion comprises the steps of:
(d′) determining the amount of a natural or non-natural amino acid which is present only in the linear peptides;
(d″) determining the amount of a natural or non-natural amino acid which is common to the library of two or more cyclic peptides and the linear peptides; and
(d′″) calculating the proportion.

9. A method for determining the amount of a linear peptide present in a cyclic peptide library comprising the steps of:
(a) determining the amount of a natural or non-natural amino acid which is present only in the linear peptide;
(b) determining the amount of a natural or non-natural amino acid which is common to the cyclic peptide library and the linear peptide; and
(c) calculating the amount of linear peptide present.

10. The composition of claim 1 which results from step (c).

11. The composition of claim 2 which results from step (a″).

12. The composition of claim 10 wherein a cyclic peptide within the library of cyclic peptides comprises physiological activity.

13. The composition of claim 12 wherein the cyclic peptide comprising physiological activity is isolated from the library.

14. The composition of claim 13 wherein the isolated cyclic peptide is the subject of an investigational new drug application or a new drug application.
